(19) United States
(12) Patent Application Publication
(10) Pub. No.: US 2022/0128593 A1
(43) Pub. Date: Apr. 28, 2022

(54) SENSING SYSTEM AND PAIRING METHOD THEREOF

(71) Applicant: Chih-Ming Wang, Taipei City (TW)

(72) Inventor: Chih-Ming Wang, Taipei City (TW)

(73) Assignee: COMPAL ELECTRONICS, INC., Taipei City (TW)

(21) Appl. No.: 17/200,919

(22) Filed: Mar. 15, 2021

(30) Foreign Application Priority Data
Oct. 22, 2020 (TW) 109136632

(51) Int. Cl. G01P 15/18; A61B 5/11; A61B 5/00

(52) U.S. Cl. CPC G01P 15/18; A61B 5/1116; A61B 2560/0214; A61B 2562/046; A61B 2562/0219; A61B 5/002

(57) ABSTRACT

A sensing system and a pairing method thereof are provided. The pairing method of the sensing system includes: respectively disposing a plurality of sensors on a plurality of parts of a tested subject; setting the tested subjected to perform at least one setting posture, and setting the sensors to respectively provide a plurality of direction information; receiving the direction information respectively provided by the sensors; and identifying the parts where the sensors are respectively arranged according to the direction information. Wherein, when the tested subject performs any one of the at least one setting posture, at least two of the direction information provided by the sensors are different.

### CROSS-REFERENCE TO RELATED APPLICATION

This application claims the priority benefit of Taiwan application serial no. 109136632, filed on Oct. 22, 2020. The entirety of the above-mentioned patent application is hereby incorporated by reference herein and made a part of this specification.

### BACKGROUND

### Field of the Disclosure

The disclosure relates to a sensing system, and particularly relates to a sensing system that can quickly complete the pairing operation with the sensor.

### Description of Related Art

For patients undergoing rehabilitation therapy, the degree of bending angles of certain parts of the body and limbs is an important evaluation index. With the advancement of today's electronic technology, electronic instruments are often adopted to reduce the inaccuracy of measurement results from human factors.

In today's technical field, multiple MEMS sensors can be disposed on the target to be measured, and can be positioned through the space orientation of the MEMS sensor. Through mathematical computation, an actual included angle between any two MEMS sensors can be obtained. Then, by calculating the mathematical trigonometric function vector projection, it is possible to accurately calculate the actual angle of the included angle in the sagittal plane, the median plane and the horizontal plane.

In order to obtain valid data, it is necessary to know where on the human body each MEMS sensor is arranged. Conventional technology often arranges specific MEMS sensors at specific parts, or manually sets the MEMS sensors arranged at different parts of the body one by one, which reduces convenience of use of MEMS sensors.

### SUMMARY OF THE DISCLOSURE

The disclosure provides a sensing system and a pairing method thereof, which can quickly perform a pairing operation of a sensor.

The pairing method of the sensing system includes: respectively disposing a plurality of sensors on a plurality of parts of a tested subject; setting the tested subject to perform at least one setting posture, and setting the sensors to respectively provide a plurality of direction information; receiving the direction information respectively provided by the sensors; and identifying the parts where the sensors are respectively disposed according to the direction information. Specifically, when the tested subject performs any one of the at least one setting posture, at least two of the direction information respectively provided by the sensors are different.

The sensing system of the disclosure includes a plurality of sensors and a detecting device. The sensor is configured for being disposed at a plurality of parts of the tested subject. The detecting device is electrically coupled to the sensor. When the tested subject is set to at least one setting posture, the sensor provides a plurality of direction information respectively. The detecting device is configured to receive the direction information respectively provided by the sensor, and identify the part where the sensors are respectively disposed according to the direction information. Specifically, when the tested subject is set to any of the at least one setting posture, at least two of the direction information respectively provided by the sensors are different.

Based on the above, in the embodiment of the disclosure, by setting the tested subject to one or more setting postures, and setting that at least two of the direction information respectively provided by the sensors are different when the tested subject is set to any of the setting postures, through identifying the difference between the direction information, it is possible to identify a plurality parts of the tested subject respectively corresponding to the plurality of sensors. In this manner, the pairing operation of the sensor can be completed quickly and automatically, thereby improving the convenience of use of sensors.

### BRIEF DESCRIPTION OF THE DRAWINGS

FIG. 1 shows a flowchart of a pairing method of a sensing system according to an embodiment of the disclosure.

FIG. 2A shows a schematic diagram of a sensing system according to an embodiment of the disclosure.

FIG. 2B is a schematic diagram of a sensing system according to another embodiment of the disclosure.

FIG. 3 is a schematic diagram of the generating method of direction vector.

FIG. 4A and FIG. 4B are schematic diagrams of a pairing method of a sensing system according to another embodiment of the disclosure.

FIG. 5 shows a flowchart of a pairing method of a sensing system according to an embodiment of the disclosure.

FIG. 6 shows a schematic diagram of a sensor according to an embodiment of the disclosure.

FIG. 7 is a schematic diagram of a sensing system according to another embodiment of the disclosure.

FIG. 8 is a schematic diagram of an implementation method of a relay according to an embodiment of the disclosure.

FIG. 9A and FIG. 9B are schematic diagrams of implementation methods of a sensor according to an embodiment of the disclosure.

### DESCRIPTION OF EMBODIMENTS

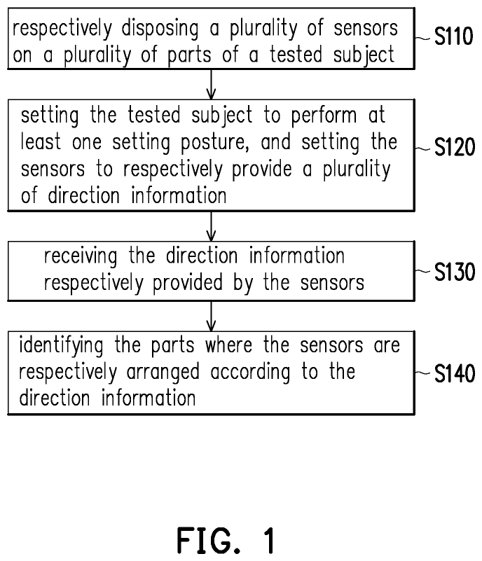

Please refer to FIG. 1, FIG. 1 shows a flowchart of a pairing method of a sensing system according to an embodiment of the disclosure. In this embodiment, the sensing system includes a detecting device and a plurality of sensors. In this embodiment, the pairing method of the sensing system is adopted to identify the positions where the sensors are disposed respectively. Specifically, in step S110, a plurality of sensors are respectively arranged at a plurality of parts of the tested subject. Then, in step S120, the tested subject is set to one or more setting postures, and the sensors are configured to respectively provide a plurality of direction information. In step S130, when the tested subject is set to a setting posture, the plurality of direction information provided by the sensors are received. Moreover, in step S140, the plurality of parts of the tested subject where the plurality of sensors are respectively disposed are identified according to the different direction information.

In this embodiment, the direction information can be expressed in the form of (but not limited to) quaternion and/or the angle between the gravity axis.

Please note here that in this embodiment, when the tested subject is set to any setting posture, it can be set that at least two of the plurality of direction information respectively provided by the plurality of sensors are different. Therefore, according to each of the setting posture set for the tested subject, it is possible to identify the parts where two or more sensors are arranged according to at least two different direction information.

In this embodiment, if the tested subject is in the first setting posture and the plurality of direction information provided by all the sensors can be different, all the parts where all of the sensors are disposed can be identified at once. If the tested subject is in the first setting posture and the directions of all of the sensors cannot be identified, the tested subject can be set to the second setting posture, and the operation of identifying the difference between direction information of the sensors can be performed. The above operation can be performed continuously until the parts where all the sensors are located are identified.

Incidentally, the plurality of sensors in the embodiment of the disclosure may all be the same electronic component, and each sensor may be arranged on any part of the tested subject.

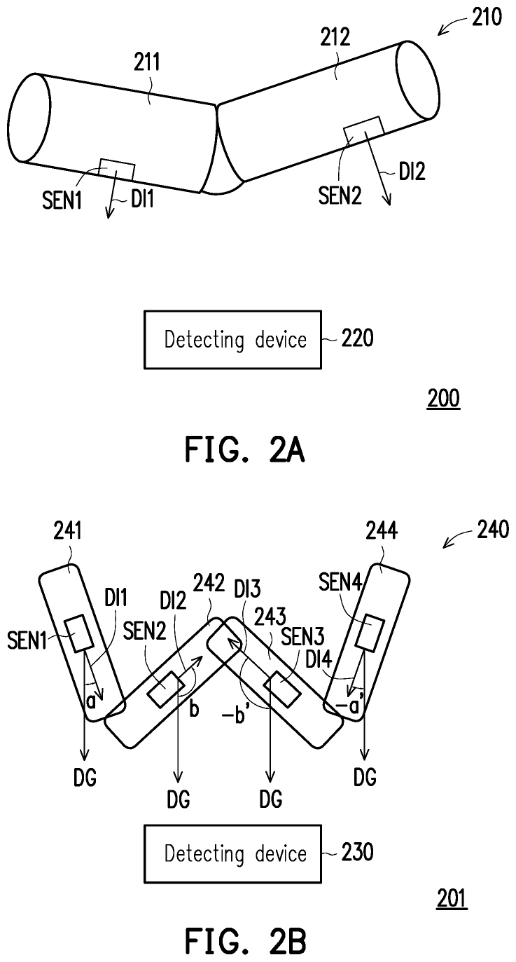

Reference may be made to FIG. 2A for the following description. FIG. 2A shows a schematic diagram of a sensing system according to an embodiment of the disclosure. The sensing system 200 includes a plurality of sensors SEN1 and SEN2 and a detecting device 220. The sensors SEN1 and SEN2 may be respectively arranged on a plurality of parts 211 and 212 of the tested subject 210. In this embodiment, there can be movable joints between the parts 211 and 212, and through adjustment, the tested subject 210 can be set to one or more setting postures. The sensors SEN1 and SEN2 are MEMS sensors. The sensors SEN1 and SEN2 respectively provide direction information DI1 and DI2 according to the inertia generated by their positions. In FIG. 2A, there is a bending angle at the distance between the parts 211 and 212 to form a setting posture, and the sensors SEN1 and SEN2 provide direction information DI1 and DI2, respectively.

The detecting device 220 is electrically connected to the sensors SEN1 and SEN2 in a wireless manner, and receives direction information DI1 and DI2. In this embodiment, when the tested subject 210 is in a setting posture, the sensors SEN1 and SEN2 provide different direction information DI1 and DI2, respectively. The detecting device 220 can determine the parts 211 and 212 where the sensors SEN1 and SEN2 are respectively arranged by identifying the difference between the direction information DI1 and DI2 and the corresponding setting posture.

In this embodiment, the detecting device 220 may be any handheld or fixed electronic device with computing capability, such as a smart phone. The smart phone can perform the pairing operation of the sensors SEN1 and SEN2 by running the application and connecting with the sensors SEN1 and SEN2. The detecting device 220 can provide a graphical user interface (GUI) for the tester to operate and complete the pairing operation of the sensors SEN1 and SEN2.

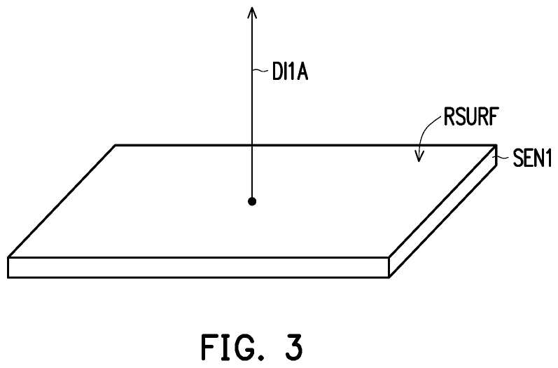

In this embodiment, taking the sensor SEN1 as an example, the direction information DI1 provided by the sensor SEN1 may include the direction vector and angle to the ground of the sensor SEN1. Specifically, referring to FIG. 3, FIG. 3 is a schematic diagram of the generating method of direction vector. The sensor SEN1 can preset a reference plane RSURF, and generate the direction vector DI1A according to the normal vector of the reference plane RSURF. Specifically, the reference plane RSURF is actually an imaginary plane, which is only used for determining the direction vector DI1A, not an actual plane.

Please refer to FIG. 2A again. In this embodiment, the detecting device 220 can receive the direction information DI1 and DI2 through a wireless transmission method such as Bluetooth. Alternatively, the detecting device 220 can also receive the direction information DI1 and DI2 through any other form of wireless communication method, and the disclosure is not limited thereto.

In addition, in the mechanism for determining the difference between the direction information DI1 and DI2, the direction information DI1 and DI2 need to be distinguishable from each other to a certain degree so that the detecting device 220 can identify that the direction information DI1 and DI2 are different. For example, based on the three-dimensional spatial coordinates, when the detecting device 220 determines that the direction information DI1 and DI2 are on different octants, the detecting device 220 can identify that the direction information DI1 and DI2 are different. Alternatively, the detecting device 220 can also calculate the angle difference between the direction information DI1 and DI2. When the angle difference is greater than a preset value, the detecting device 220 can identify that the direction information DI1 and DI2 are different.

Referring to FIG. 2B below, FIG. 2B is a schematic diagram of a sensing system according to another embodiment of the disclosure. The sensing system 201 includes a plurality of sensors SEN1 to SEN4 and a detecting device 230. The sensors SEN1 to SEN2 can be respectively arranged on multiple parts 241 to 244 of the tested subject 240. The sensors SEN1 to SEN2 respectively provide direction information DI1 to DI4 according to the inertia generated by their positions. Specifically, in the sensing system of this embodiment, before the actual detecting action is performed, for a setting posture, the angle between the direction information DI1 and a reference vector DG can be set as a; the angle between the direction information DI2 and the reference vector DG can be set as b; the angle between the direction information DI3 and the reference vector DG can be set as −b′; and the angle between the direction information DI4 and the reference vector DG can be set as −a′. The detecting device 230 can add a tolerance value (for example, plus or minus 20 degrees) to the angles a, b, −b′ and −a′, and can obtain four preset ranges, including 25 degrees to 65 degrees, 115 degrees to 155 degrees, −155 degrees to −115 degrees, and −65 degrees to −25 degrees respectively. Specifically, the reference vector DG is generated according to the direction of the sensors SEN1 to SEN4 to the ground.

During the actual detecting operation, the detecting device 230 can receive the direction information DI1 to DI4 respectively transmitted by the sensors SEN1 to SEN4 through wireless transmission, and obtain the multiple angles x, y, z and w mentioned above. Setting the angles x, y, z, and w as 40 degrees, 150 degrees, −100 degrees, and −55 degrees as an example, it can be determined which preset range that the direction information DI1 to D14 provided by each of the sensors SEN1 to SEN4 falls into. In this way, the corresponding part where the sensor SEN1 to SEN4 are disposed can be identified.

In this embodiment, the parts 241 and 242 may be connected to each other, and the parts 243 and 244 may also be connected to each other. In addition, the parts 241 and 242 may be a person's first upper arm and a first forearm, respectively, and the parts 243 and 244 may be a person's second forearm and a second upper arm, respectively. However, the disclosure is not limited thereto, and in other embodiments, the sensors SEN1 to SEN4 may also be arranged in non-connected parts.

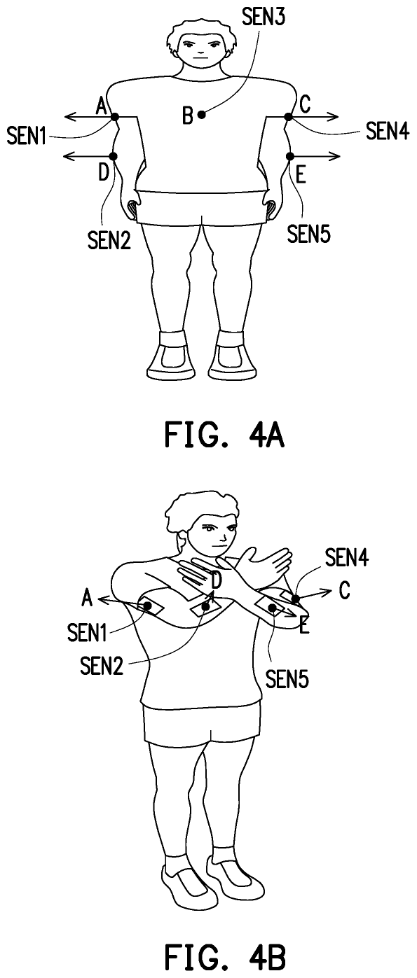

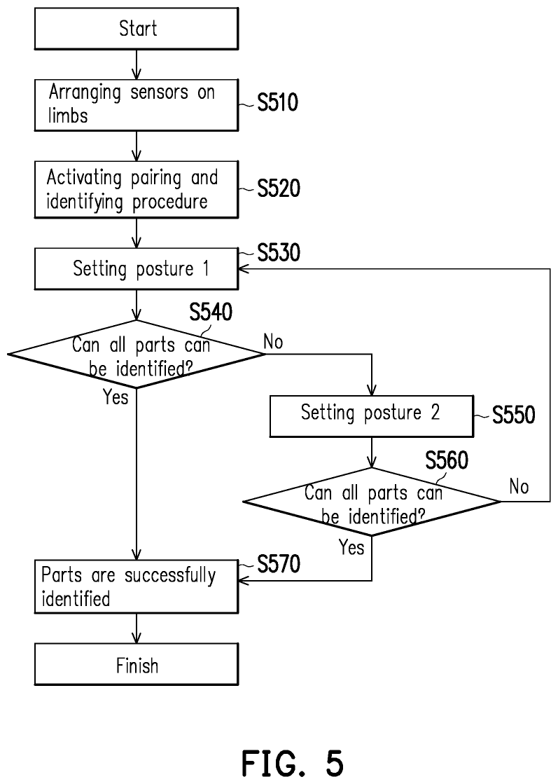

Please refer to FIG. 4A, FIG. 4B, and FIG. 5 below, wherein FIG. 4A and FIG. 4B are schematic diagrams of a pairing method of a sensing system according to another embodiment of the disclosure, FIG. 5 shows a flowchart of a pairing method of a sensing system according to an embodiment of the disclosure. In step S510, the sensors SEN1 to SENS are respectively arranged in the limbs of the human body (the tested subject). The sensor SEN1 is arranged on the upper arm of the first hand (right hand); the sensor SEN2 is arranged on the forearm of the first hand; the sensor SEN3 is arranged at the center of the human chest; the sensor SEN4 is arranged on the upper arm of the second hand (left hand); and the sensor SEN5 is arranged on the forearm of the second hand (left hand).

In step S520, the pairing identification program is activated, and in step S530, the human body is set to the setting posture 1 (the upright posture shown in FIG. 4A). In the meantime, the sensors SEN1 to SEN5 provide direction information A, D, B, C, and E respectively.

In step S540, it is determined whether all the sensors SEN1 to SEN5 on all parts can be identified. Specifically, in FIG. 4A, the direction information A and D are similar and cannot be effectively distinguished, and the direction information C and E are similar and cannot be effectively distinguished. Only the sensors SEN1 and SEN2 is the first group that can be distinguished, the sensor SEN3 is the second group that can be distinguished, and the sensors SEN4 and SEN5 are the third group that can be distinguished.

Under the condition that not all the parts where all the sensors SEN1 to SEN5 can be identified, step S550 can be performed, and the human body is set to the setting posture 2, as shown in FIG. 4B. In FIG. 4B, the direction information A, D, B, C, and E respectively provided by the sensors SEN1 to SEN5 are different, and can be effectively distinguished. Therefore, in step 5560, it is determined that all the parts where all the sensors SEN1 to SEN5 are arranged can be identified, then step S570 can be performed to determine that the position identification is successful, and the procedure is ended.

Please note here that if it is determined in step S560 that all the parts where all the sensors SEN1 to SEN5 are arranged still cannot be identified, then the human body can be set to another setting posture (setting posture 3) or reset to the setting posture 1, and further identification operation is performed. If it has been determined in step S540 that all the parts where the sensors SEN1 to SEN5 are arranged have been identified, step S570 can be directly performed and the procedure is ended.

Based on the structural difference of human body, the setting posture in this embodiment can be set according to the state of each human body. For example, when the tested human body of which the elbow joint cannot be bent overly due to some specific reasons, the posture can be set as raising the upper arm or any movement that the tested human body can complete. The point is that the tested body can set the posture so that the sensor can generate effectively distinguishable direction information, that is, the operation of identifying the parts where the sensors are located can be completed.

Although the sensors SEN1 to SEN5 in FIG. 4A and FIG. 4B are all arranged on the upper limbs of the human body, in other embodiments of the disclosure, the sensors may also be arranged on the lower limbs of the human body. The arrangement of the sensors SEN1 to SEN5 in FIG. 4A and FIG. 4B is only an example for illustration, and is not intended to limit the scope of the disclosure.

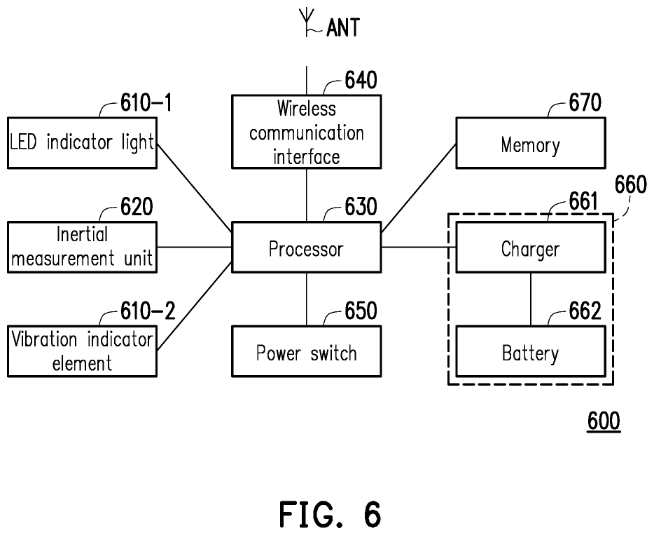

Please refer to FIG. 6, FIG. 6 shows a schematic diagram of a sensor according to an embodiment of the disclosure. The sensor 600 includes an indicator element formed by a light emitting diode (LED) indicator light 610-1 and a vibration indicator element 610-2, an inertial measurement unit (IMU) 620, a processor 630, a wireless communication interface 640, a power switch 650, a power supply element 660 and a memory 670. The IMU 620 is configured to generate multi-axis information (for example, 6-axis information) according to the inertial state of the sensor 600. The processor 630 is coupled to the IMU 620 for performing coordinate conversion operations on the multi-axis information to generate direction information. The wireless communication interface 640 is coupled to the processor 630 for transmitting direction information through the antenna ANT.

In addition, the memory 670 can be configured to store setting data and/or temporary data required by the processor 630. The memory 670 can be any form of memory, and the disclosure is not limited thereto. The power supply element 660 includes a battery 662 and a charger 661. The power supply element 660 can provide the operating power required by the sensor 600 through the battery 662. The battery 662 can be a rechargeable battery, and can be charged or discharged through the charge and discharge management operations performed by the charger 661.

The power switch 650 is coupled to the processor 630 and configured to control whether the sensor 600 is activated or not. When the sensor 600 is activated, the processor 630 can activate the detecting action of the IMU, and transmit direction information through the wireless communication interface 640. In this embodiment, the wireless communication interface 640 may be a Bluetooth communication interface, or may also be another form of wireless communication interface, and the disclosure is not limited thereto.

The LED indicator light 610-1 and the vibration indicator element 610-2 perform the function of indicating the working state of the sensor 600 through the means of light energy and kinetic energy, respectively. Certainly, in other embodiments of the disclosure, a buzzer can also be provided, and a sound wave can be used to perform an indicating action, and the disclosure is not limited thereto.

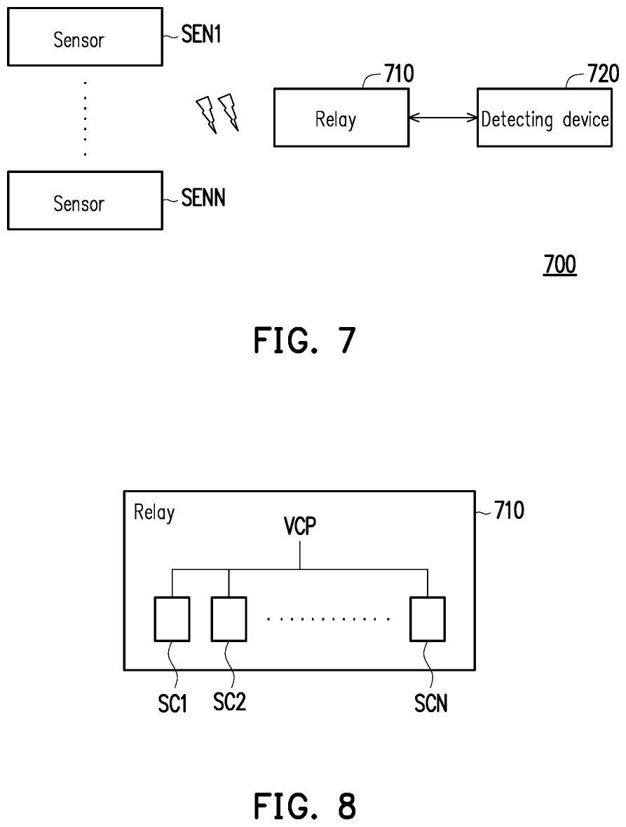

Please refer to FIG. 7 below. FIG. 7 is a schematic diagram of a sensing system according to another embodiment of the disclosure. The sensing system 700 includes a plurality of sensors SEN1 to SENN, a relay 710 and a detecting device 720. The relay 710 is electrically coupled between the sensors SEN1 to SENN and the detecting device 720. The relay 710 can serve as a relay station for transmitting the direction information provided by the sensors SEN1 to SENN. The relay 710 receives the direction information provided by the sensors SEN1 to SENN through wireless communication, and then forwards the direction information to the detecting device 720.

In this embodiment, the direction information can be transmitted between the relay 710 and the detecting device 720 through wired or wireless communication. In some embodiments of the disclosure, the detecting device 720 can perform the pairing operation of the sensors SEN1 to SENN through the relay 710 by means of remote monitoring.

Please refer to FIG. 7 and FIG. 8 both. FIG. 8 is a schematic diagram of an implementation method of a relay according to an embodiment of the disclosure. In FIG. 8, multiple accommodating spaces SC1 to SCN can be provided on the relay 710. When the sensing system 700 is in an idle state, the relay 710 can provide the accommodating spaces SC1 to SCN to accommodate the sensors SEN1 to SENN respectively. In the meantime, the relay 710 can provide the charging power VCP to the accommodating spaces SC1 to SCN, and perform charging actions on the sensors SEN1 to SENN.

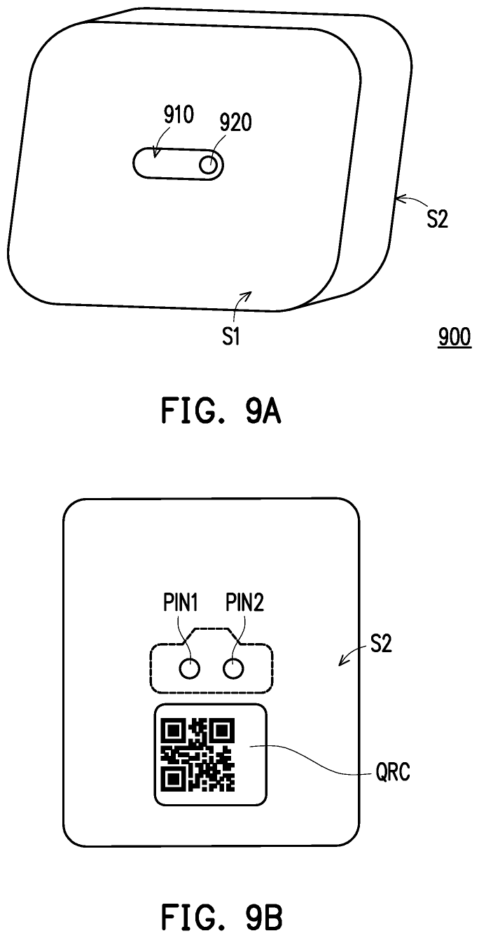

Please refer to FIG. 9A and FIG. 9B. FIG. 9A and FIG. 9B are schematic diagrams of implementation methods of a sensor according to an embodiment of the disclosure. The sensor 900 has a first surface S1 and a second surface S2 opposite to each other. A power switch 910 and an LED indicator light 920 can be provided on the first surface S1. By pressing the power switch 910, the sensor 900 can be turned on, and the sensor 900 can be operated with a vibration indication. The LED indicator light 920 can be correspondingly turned on to inform the user that the sensor 900 is currently operating normally. The LED indicator light 920 can also indicate the working condition of the sensor 900 through the displayed color. For example, a green light indicates that the sensor 900 is working normally, and a red light indicates that the sensor 900 is malfunctioned or has insufficient voltage.

In FIG. 9B, power pins PIN1 and PIN2 may be provided on the second surface of the sensor 900. When the sensor 900 is set in the accommodating space of the relay, the sensor 900 can receive charging power supply through the power pins PIN1 and PIN2. In this way, the sensor 900 can be charged according to the charging power supply. In addition, an identification code QRC is also provided on the second surface of the sensor 900. In this embodiment, the identification code information QRC may be a quick response code (QR code) or any other identification pattern. The identification code information QRC is provided for the detecting device to perform a scanning operation, and accordingly perform a pairing operation between the detecting device and the sensor 900.

In summary, the disclosure sets the tested subject to one or more preset postures. Through the preset postures, the plurality of sensors arranged on the tested subject provide a plurality of direction information among which at least two are different. In this manner, according to the different direction information, the parts where the sensors are located can be identified, and the pairing operation on the sensors can be quickly completed.

What is claimed is:

1. A pairing method of a sensing system, comprising:
disposing a plurality of sensors on a plurality of parts of a tested subject;
setting the tested subject to at least one setting posture, and setting the plurality of sensors to respectively provide a plurality of direction information;
receiving the plurality of direction information respectively provided by the plurality of sensors; and
identifying the plurality of parts where the plurality of sensors are respectively disposed according to the plurality of direction information,
wherein when the tested subject is in any one of the at least one setting posture, at least two of the plurality of direction information respectively provided by the plurality of sensors are different.

2. The pairing method according to claim 1, wherein each of plurality of the direction information comprises a direction vector and an angle to ground corresponding to each of the plurality of sensors.

3. The pairing method according to claim 1, wherein the step of setting the plurality of sensors to respectively provide the plurality of direction information comprises:
generating multi-axis information by each of the plurality of sensors through an inertial measurement unit; and
performing a coordinate conversion operation on the multi-axis information to generate each of the plurality of direction information.

4. The pairing method according to claim 1, wherein the step of receiving the plurality of direction information respectively provided by the plurality of sensors comprises:
providing a detecting device to receive the plurality of direction information through a wireless communication interface.

5. The pairing method according to claim 4, wherein the step of identifying the plurality of parts where the plurality of sensors are respectively arranged according to the plurality of direction information further comprises:
when the tested subject is in a first setting posture, the detecting device is provided to find out a plurality of different first direction information, and find out the plurality of parts respectively corresponding to a plurality of first sensors respectively corresponding to the plurality of first direction information;
providing the detecting device to determine whether there are a plurality of second direction information that are the same; and
when the detecting device determines that there are the plurality of identical second direction information, the detecting device is set to send a prompt signal, and the tested subject is changed to a second setting posture.

6. The pairing method according to claim 5, further comprising:
when the tested subject is in the second setting posture, the detecting device is provided to find out a plurality of different third direction information, and find out the plurality of parts respectively corresponding to a plurality of second sensors respectively corresponding to the plurality of third direction information.

7. A sensing system, comprising:
a plurality of sensors configured on a plurality of parts of a tested subject; and
a detecting device electrically coupled to the plurality of sensors,
wherein when the tested subject is set to at least one setting posture, for a plurality of direction information provided by the plurality of sensors respectively, the detecting device is configured to:
receive the plurality of direction information provided by the plurality of sensors respectively, and identify the plurality of parts where the plurality of sensors are respectively arranged according to the plurality of direction information,
wherein, when the tested subject is in any one of the at least one setting posture, at least two of the plurality of direction information respectively provided by the plurality of sensors are different.

8. The sensing system according to claim 7, wherein each of the plurality of sensors generates each of the plurality of corresponding direction information according to a direction vector and an angle to ground.

9. The sensing system according to claim 7, wherein each of the plurality of sensors comprises:
an inertial measurement unit configured to generate multi-axis information;
a processor, coupled to the inertial measurement unit, performing a coordinate conversion operation on the multi-axis information to generate each of the plurality of direction information; and
a wireless communication interface, coupled to the processor, and configured to send each of the plurality of direction information.

10. The sensing system according to claim 9, wherein each of the plurality of sensors further comprises:
an indicator element for performing an indicating operation indicating a working state of each of the plurality of sensors;
a power supply element for providing an operating power supply for each of the plurality of sensors; and
a memory coupled to the processor.

11. The sensing system according to claim 7, further comprising:
a relay, coupled between the plurality pf sensors and the detecting device, configured for transmitting the plurality of direction information to the detecting device.

12. The sensing system according to claim 11, wherein the relay comprises a plurality of accommodating spaces for accommodating the plurality of sensors when the sensing system is in an idle state.

13. The sensing system according to claim 12, wherein the relay provides a charging power supply to the plurality of accommodating spaces to charge the plurality of sensors.

14. The sensing system according to claim 7, wherein the detecting device is configured to:
when the tested subject is in a first setting posture, find out a plurality of different first direction information, and find out the plurality of parts respectively corresponding to a plurality of first sensors respectively corresponding to the plurality of first direction information;
determining whether there are a plurality of identical second direction information; and
when the detecting device determines that there are the plurality of identical second direction information, send a prompt signal, and change the tested subject to a second setting posture.
